(19) United States
(12) Patent Application Publication
(10) Pub. No.: US 2026/0170805 A1
(43) Pub. Date: Jun. 18, 2026

(54) IMAGE CLASSIFICATION METHOD, AND METHOD AND APPARATUS FOR TRAINING IMAGE CLASSIFICATION MODEL

(71) Applicant: Alibaba (China) Co., Ltd., Hangzhou (CN)

(72) Inventors: Yingda XIA, Washington, DC (US); Mingze YUAN, Beijing (CN); Ling ZHANG, Washington, DC (US); Jiawen YAO, Beijing (CN); Mingyan QIU, Hangzhou (CN); Hexin DONG, Beijing (CN); Jingren ZHOU, Bellevue, WA (US); Le LU, Bethesda, MD (US)

(21) Appl. No.: 19/368,870

(22) Filed: Oct. 24, 2025

Related U.S. Application Data
(63) Continuation of application No. PCT/CN2024/103726, filed on Jul. 4, 2024.

(30) Foreign Application Priority Data
Jul. 4, 2023 (CN) 202310822213.1

(51) Int. Cl. G06V 10/764; G06T 7/00; G06V 10/26; G06V 10/52; G06V 10/762; G06V 10/774; G06V 10/82; G16H 50/20

(52) U.S. Cl. CPC G06V 10/764; G06T 7/0012; G06V 10/26; G06V 10/52; G06V 10/762; G06V 10/774; G06V 10/82; G06T 2207/10081; G06T 2207/20081; G06T 2207/20084; G06T 2207/30092; G06T 2207/30096; G06V 2201/031; G16H 50/20

(57) ABSTRACT

Embodiments of this application disclose an image classification method, and a method and an apparatus for training an image classification model. A main technical solution includes: obtaining a to-be-classified image; performing feature extraction on the to-be-classified image to obtain a feature representation of the image; performing, by using the feature representation of the image, cross-attention processing on an initial representation of a plurality of cluster centers obtained through pre-training to obtain a plurality of cluster center representations; performing decoding by using the feature representation of the image and the plurality of cluster center representations to obtain a category assignment matrix; and performing classification by using the plurality of cluster center representations and the category assignment matrix to obtain a classification result indicating whether the to-be-classified image belongs to a target category. According to this application, an image classification result can have a higher accuracy rate.

### CROSS-REFERENCE TO RELATED APPLICATION(S)

This application is a continuation application of PCT/CN2024/103726, filed on Jul. 4, 2024, which claims priority to Chinese Patent Application No. 202310822213.1, filed with the China National Intellectual Property Administration on Jul. 4, 2023 and entitled “IMAGE CLASSIFICATION METHOD, AND METHOD AND APPARATUS FOR TRAINING IMAGE CLASSIFICATION MODEL”, which are each incorporated herein by reference in its entirety.

### TECHNICAL FIELD

This application relates to the field of computer vision technologies, and in particular, to an image classification method, and a method and an apparatus for training an image classification model.

### BACKGROUND

Image classification is to distinguish between images of different categories according to semantic information of the images, and is an important basic problem in computer vision. The image classification is widely applied to a large quantity of fields, for example, traffic scene recognition in the field of transportation, automatic classification of commodity images in the field of e-commerce, and image recognition in the field of medicine.

In some special fields, there is a high requirement on an accuracy rate and a recall rate of image classification. Although currently there is a related technology of classifying images by using a deep learning model, an accuracy rate of a classification result still remains to be improved.

### SUMMARY

This application provides an image classification method, and a method and an apparatus for training an image classification model, which, among others, improves an accuracy rate of an image classification result.

Embodiments of this application provide the following solutions.

According to a first aspect, an image classification method is provided, including: obtaining a to-be-classified image;
performing feature extraction on the to-be-classified image, to obtain a feature representation of the image;
performing, by using the feature representation of the image, cross-attention processing on an initial representation of a plurality of cluster centers obtained through pre-training, to obtain a plurality of cluster center representations;
performing decoding by using the feature representation of the image and the plurality of cluster center representations, to obtain a category assignment matrix; and
performing classification by using the plurality of cluster center representations and the category assignment matrix, to obtain a classification result indicating whether the to-be-classified image belongs to a target category.

According to a possible implementation of the embodiments of this application, the method further includes:
segmenting the to-be-classified image by using the category assignment matrix, to obtain an image area of a preset category, where the preset category includes the target category.

According to a possible implementation of the embodiments of this application, the performing feature extraction on the to-be-classified image, to obtain a feature representation of the image includes:
performing feature extraction on the to-be-classified image, to obtain a feature representation of each element Token at a plurality of resolutions, and determining a feature representation of each Token at a highest resolution as the feature representation of the image.

According to a possible implementation of the embodiments of this application, the performing, by using the feature representation of the image, cross-attention processing on an initial representation of a plurality of cluster centers obtained through pre-training includes:
obtaining a query matrix by using the initial representation of the plurality of cluster centers, inputting the query matrix to a multilayer concatenated transformer network, where each layer of Transformer network is in one-to-one correspondence with each resolution based on an ascending order of resolutions, obtaining, by each layer of Transformer network, a key matrix and a value matrix by using a feature representation of each Token at a corresponding resolution, and performing cross-attention processing on the query matrix input to the layer of Transformer network, to obtain a query matrix output by the layer of Transformer network; and
obtaining the plurality of cluster center representations by using a query matrix output by a last layer of Transformer network.

According to a possible implementation of the embodiments of this application, the performing classification by using the plurality of cluster center representations and the category assignment matrix includes:
performing averaging on the plurality of cluster center representations, to obtain a cluster-averaged representation;
performing pooling on the category assignment matrix, to obtain a cluster-pooled feature; and
integrating the cluster-averaged representation and the cluster-pooled representation, and performing classification by using a feature representation obtained through integration, to obtain the classification result indicating whether the to-be-classified image belongs to the target category.

According to a second aspect, a method for training an image classification model is provided, including:
obtaining training data including a plurality of training samples, where the training sample includes an image sample and a label indicating whether the image sample belongs to a target category; and
training an image classification model by using the training data, where the image classification model includes: a feature extraction network, a first decoding network, a second decoding network, and a classification network; the feature extraction network performs feature extraction on the image sample, to obtain a feature representation of the image sample; the first decoding network performs cross-attention processing on an initial representation of a plurality of cluster centers by using the feature representation of the image sample, to obtain a plurality of cluster center representations; the second decoding network performs decoding by using the feature representation of the image sample and the plurality of cluster center representations, to obtain a category assignment matrix; the classification network performs classification by using the plurality of cluster center representations and the category assignment matrix, to obtain a classification result indicating whether the image sample belongs to a target category; and a target of the training includes: minimizing a difference between the classification result and a corresponding label.

According to a possible implementation of the embodiments of this application, the training sample further includes an area mask of a preset category marked on the image sample; and the image classification model further includes a segmentation network;
the segmentation network segments the image sample by using the category assignment matrix, to obtain an image area of the preset category, where the preset category includes the target category; and
the target of the training further includes: minimizing a difference between the image area of the preset category and a corresponding area mask.

According to a possible implementation of the embodiments of this application, that the feature extraction network performs feature extraction on the image sample, to obtain a feature representation of the image sample includes: performing feature extraction on the image sample, to sequentially obtain a feature representation of each element Token at a plurality of resolutions, and determining a feature representation of each Token at a highest resolution as the feature representation of the image sample; and
the first decoding network includes a multilayer concatenated Transformer network, the initial representation of the plurality of cluster centers is determined as a query matrix input to a first layer of Transformer network, each layer of Transformer network is in one-to-one correspondence with each resolution based on an ascending order of resolutions, each layer of Transformer network obtains a key matrix and a value matrix by using a feature representation of each Token at a corresponding resolution, and cross-attention processing is performed on the query matrix input to the layer of Transformer network, to obtain a query matrix output by the layer of Transformer network; and the plurality of cluster center representations are obtained by using a query matrix output by a last layer of Transformer network.

According to a possible implementation of the embodiments of this application, that the classification network performs classification by using the plurality of cluster center representations and the category assignment matrix includes:
performing, by the classification network, averaging on the plurality of cluster center representations, to obtain a cluster-averaged representation;
performing pooling on the category assignment matrix, to obtain a cluster-pooled representation; and
integrating the cluster-averaged representation and the cluster-pooled representation, and performing classification by using a feature representation obtained through integration, to obtain the classification result indicating whether the image sample belongs to the target category.

According to a third aspect, an image classification method is provided, performed by a cloud server, and including:
obtaining a to-be-classified image from a user terminal;
performing feature extraction on the to-be-classified image, to obtain a feature representation of the image;
performing, by using the feature representation of the image, cross-attention processing on an initial representation of a plurality of cluster centers obtained through pre-training, to obtain a plurality of cluster center representations;
performing decoding by using the feature representation of the image and the plurality of cluster center representations, to obtain a category assignment matrix; performing classification by using the plurality of cluster center representations and the category assignment matrix, to obtain a classification result indicating whether the to-be-classified image belongs to a target category; and
returning the classification result to the user terminal.

According to a fourth aspect, a computer-aided diagnosis method for a cancer is provided, including:
obtaining a medical image acquired for a target organ;
performing feature extraction on the medical image, to obtain a feature representation of the medical image;
performing, by using the feature representation of the medical image, cross-attention processing on an initial representation of a plurality of cluster centers obtained through pre-training, to obtain a plurality of cluster center representations;
performing decoding by using the feature representation of the medical image and the plurality of cluster center representations, to obtain a category assignment matrix; and
performing classification by using the plurality of cluster center representations and the category assignment matrix, to obtain a classification result indicating whether the medical image belongs to a target lesion.

According to a fifth aspect, a computer-aided diagnosis method for a stomach cancer is provided, including:
obtaining a medical image acquired for a stomach;
performing feature extraction on the medical image, to obtain a feature representation of the medical image;
performing, by using the feature representation of the medical image, cross-attention processing on an initial representation of a plurality of cluster centers obtained through pre-training, to obtain a plurality of cluster center representations;
performing decoding by using the feature representation of the medical image and the plurality of cluster center representations, to obtain a category assignment matrix; and
performing classification by using the plurality of cluster center representations and the category assignment matrix, to obtain a classification result indicating whether the medical image belongs to a stomach cancer.

According to a sixth aspect, a computer-aided diagnosis system for a cancer is provided, including a memory, a processor, and a computer program stored in the memory and run on the processor, where the processor, when executing the computer program, is operable to perform a computer-aided diagnosis method for a cancer, and the method includes:
obtaining a medical image acquired for a target organ;
performing feature extraction on the medical image, to obtain a feature representation of the medical image;
performing, by using the feature representation of the medical image, cross-attention processing on an initial representation of a plurality of cluster centers obtained through pre-training, to obtain a plurality of cluster center representations;
performing decoding by using the feature representation of the medical image and the plurality of cluster center representations, to obtain a category assignment matrix; and
performing classification by using the plurality of cluster center representations and the category assignment matrix, to obtain a classification result indicating whether the medical image belongs to a target lesion.

According to a seventh aspect, an image classification apparatus is provided, including:
an image obtaining unit, configured to obtain a to-be-classified image;
a feature extraction unit, configured to perform feature extraction on the to-be-classified image, to obtain a feature representation of the image;
a first decoding unit, configured to perform, by using the feature representation of the image, cross-attention processing on an initial representation of a plurality of cluster centers obtained through pre-training, to obtain a plurality of cluster center representations;
a second decoding unit, configured to perform decoding by using the feature representation of the image and the plurality of cluster center representations, to obtain a category assignment matrix; and
an image classification unit, configured to perform classification by using the plurality of cluster center representations and the category assignment matrix, to obtain a classification result indicating whether the to-be-classified image belongs to a target category.

According to an eighth aspect, an apparatus for training an image classification model is provided, including:
a sample obtaining unit, configured to obtain training data including a plurality of training samples, where the training sample includes an image sample and a label indicating whether the image sample belongs to a target category; and
a model training unit, configured to train an image classification model by using the training data, where the image classification model includes: a feature extraction network, a first decoding network, a second decoding network, and a classification network; the feature extraction network performs feature extraction on the image sample, to obtain a feature representation of the image sample; the first decoding network performs cross-attention processing on an initial representation of a plurality of cluster centers by using the feature representation of the image sample, to obtain a plurality of cluster center representations; the second decoding network performs decoding by using the feature representation of the image sample and the plurality of cluster center representations, to obtain a category assignment matrix; the classification network performs classification by using the plurality of cluster center representations and the category assignment matrix, to obtain a classification result indicating whether the image sample belongs to a target category; and a target of the training includes: minimizing a difference between the classification result and a corresponding label.

According to a ninth aspect, an image classification apparatus is provided, including:
an image obtaining unit, configured to obtain a medical image acquired for a target organ;
a feature extraction unit, configured to perform feature extraction on the medical image, to obtain a feature representation of the medical image;
a first decoding unit, configured to perform, by using the feature representation of the medical image, cross-attention processing on an initial representation of a plurality of cluster centers obtained through pre-training, to obtain a plurality of cluster center representations;
a second decoding unit, configured to perform decoding by using the feature representation of the medical image and the plurality of cluster center representations, to obtain a category assignment matrix; and
an image classification unit, configured to perform classification by using the plurality of cluster center representations and the category assignment matrix, to obtain a classification result indicating whether the medical image belongs to a target lesion. According to a tenth aspect, a computer-readable storage medium is provided. The computer-readable storage medium stores a computer program. The program, when executed by a processor, implements steps of the method according to any one of the first aspect to the fifth aspect.

According to an eleventh aspect, an electronic device is provided, including:
one or more processors; and
a memory associated with the one or more processors, where the memory is configured to store program instructions, and the program instructions, when read and executed by the one or more processors, implement steps of the method according to any one of the first aspect to the fifth aspect.

According to a twelfth aspect, a computer program is provided. The computer program, when executed by a computer, causes the computer to perform steps of the method according to any one of the first aspect to the fifth aspect.

According to a thirteenth aspect, a computer program product is provided, including a computer program. The computer program, when executed by a computer, causes the computer to perform steps of the method according to any one of the first aspect to the fifth aspect.

According to the specific embodiments provided by this application, this application discloses the following technical effects.
(1) In this application, high-level semantics included in the plurality of cluster centers obtained through pre-training are jointly decoded with the feature representation of the image, to obtain the category assignment matrix, so that the category assignment matrix can reflect an image feature associated with the cluster center, e.g., the image feature is matched to a corresponding cluster center, thereby enabling an image classification result obtained based on the cluster center representation and the category assignment matrix to have a higher accuracy rate and recall rate.
(2) In this application, the to-be-classified image can be further segmented by using the category assignment matrix, to obtain the image area of the preset category, to provide interpretable reference for image classification.
(3) In this application, a multi-resolution feature is extracted, and cross-attention processing is performed by using the multi-resolution feature and the initial representation of the plurality of cluster centers, so that an image texture can be perceived from a plurality of scales in a process of obtaining the plurality of cluster center representations. In addition, the feature representation of the plurality of cluster centers and the feature representation of the image are re-assigned, so that a classification process and a segmentation process are sensitive to local semantics, and have global awareness, thereby further improving an accuracy rate and a recall rate of image classification and image segmentation.
(4) In a process of training the image classification model, auxiliary training can be performed on the image classification model by using a difference between the image area of the preset category obtained by segmenting the image sample by using the segmentation network and the area mask that is of the preset category and that is marked for the image sample, thereby further improving an effect and performance of the image classification model.

Certainly, any product implementing this application does not necessarily need to achieve all the advantages described above at the same time.

### BRIEF DESCRIPTION OF THE DRAWINGS

To describe technical solutions in embodiments of this application or the related art more clearly, the following briefly introduces the accompanying drawings required for describing the embodiments. It is clear that the accompanying drawings in the following descriptions are merely some embodiments of this application, and a person of ordinary skill in the art may still derive other drawings from these accompanying drawings without creative efforts.

FIG. 1 is a diagram of a system architecture to which an embodiment of this application is applicable;

FIG. 2 is a flowchart of an image classification method according to an embodiment of this application;

FIG. 3 is a schematic diagram of a principle of an image classification model according to an embodiment of this application;

FIG. 4 is a flowchart of a method for training an image classification model according to an embodiment of this application;

FIG. 5 is a schematic diagram of a principle of training an image classification model according to an embodiment of this application;

FIG. 6 is a flowchart of an image classification method applied to the medical field according to an embodiment of this application;

FIG. 7 is a schematic block diagram of an image classification apparatus according to an embodiment of this application;

FIG. 8 is a schematic block diagram of an apparatus for training an image classification model according to an embodiment of this application; and

FIG. 9 is a schematic block diagram of an electronic device according to an embodiment of this application.

### DETAILED DESCRIPTION

Technical solutions of embodiments of this application are described below clearly and comprehensively in conjunction with accompanying drawings of the embodiments of this application. It is clear that the embodiments described are merely some rather than all of the embodiments of this application. Based on the embodiments of this application, all other embodiments obtained by a person of ordinary skill in the art fall within the protection scope of this application.

The terms used in the embodiments of the present specification are merely for describing specific embodiments, but are not intended to limit the present specification. The terms “a”, “an”, and “the” of singular forms used in the embodiments of the present specification and the appended claims are also intended to include a plurality of forms, unless the context clearly indicates otherwise.

It should be understood that the term “and/or” used herein describes only an association relationship between associated objects and represents that three relationships may exist. For example, A and/or B may represent the following three cases: only A exists, both A and B exist, and only B exists. In addition, the character “/” herein generally represents that associated objects in the context are in an “or” relationship.

Depending on a context, the term “if” used herein may be interpreted as “when . . . ” or “upon . . . ” or “in response to determining that” or “in response to detecting that”. Similarly, depending on a context, the phrase “if determining” or “if detecting (a stated action or event)” may be interpreted as “when determining” or “in response to determining”, or “when detecting (a stated action or event)” or “in response to detecting (a stated action or event)”.

In a conventional image classification method based on a deep neural network, usually, after a feature representation of an image is extracted from the image, classification is directly performed by using the extracted feature representation of the image. However, an accuracy rate and a recall rate of an image classification result obtained in some complex scenarios are low in this manner, and cannot satisfy an actual scenario requirement.

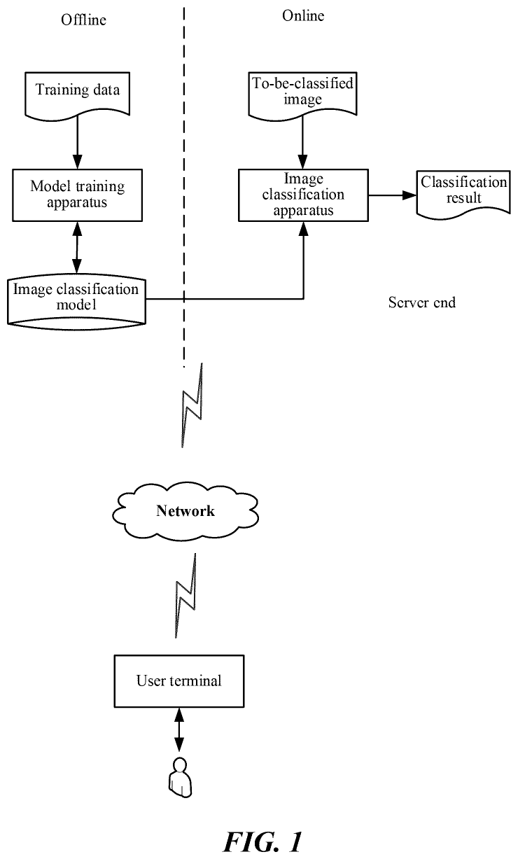

This application provides a new solution of image classification. To facilitate understanding of this application, a system architecture to which this application is applicable is first briefly described. FIG. 1 shows an example system architecture to which an embodiment of this application may be applied. As shown in FIG. 1, the system architecture includes a model training apparatus and an image classification apparatus on a server side, and may further include a user terminal communicatively coupled to the server.

The model training apparatus is configured to perform model training at before, during or after an image classification task is performed. With training data is obtained in various manners, model training may be performed by using the method provided in embodiments of this application, to obtain an image classification model.

The image classification apparatus is configured to classify a to-be-classified image by using the image classification model that has been obtained through training, to obtain a classification result, e.g., indicating whether the image belongs to a target category.

The model training apparatus and the image classification apparatus may be separately disposed as independent servers, or may be disposed in a same server or server group, or may be disposed in independent cloud servers or a same cloud server. The cloud server, also referred to as a cloud computing server or a cloud host, is a host node in a cloud computing system, to improves resource management and service scalability over physical hosts and virtual private servers (VPs, Virtual Private Server). The model training apparatus and the image segmentation apparatus may also be disposed in a computer terminal having a strong computing capability.

In a possible implementation, a user may send, by using the user terminal, the to-be-classified image to the image classification apparatus on the server side through, e.g., a network. After classifying the to-be-classified image by using the method provided in embodiments of this application, the image classification apparatus returns the classification result to the user terminal.

The user terminal may include, but is not limited to, a smart mobile terminal, a smart household, a wearable device, a smart medical device, a PC (Personal Computer, personal computer), and the like. The smart mobile device may include, for example, a mobile phone, a tablet computer, a notebook computer, a PDA (Personal Digital Assistant, personal digital assistant), and a connected car. The smart household may include, for example, a smart television and a smart refrigerator. The wearable device may include, for example, a smart watch, smart glasses, a smart band, a VR (Virtual Reality, virtual reality) device, an AR (augmented reality) device, and a mixed reality device (e.g., a device that can support virtual reality and augmented reality).

It should be noted that, in addition to performing image classification online, the image classification apparatus may perform image classification in an offline manner, for example, separately performing image classification on a batch of to-be-classified images.

It should be understood that quantities of the model training apparatuses, the image classification apparatuses, the image classification models, and the user terminals in FIG. 1 are merely illustrative examples. Based on an implementation requirement, there may be any quantity of model training apparatuses, image classification apparatuses, image classification models, and user terminals.

It should be noted that, in the present disclosure, limitations such as “first” and “second” are not limited in aspects such as a size, a sequence, and a quantity, and are merely used for distinguishing between objects in terms of names. For example, “a first image segmentation model” and “a second image segmentation model” are used for distinguishing between two models in terms of names. For another example, “a first segmentation result” and “a second segmentation result” are used for distinguishing between two segmentation results in terms of names. For another example, “a first target” and “a second target” are used for distinguishing between two targets in terms of names, and the like.

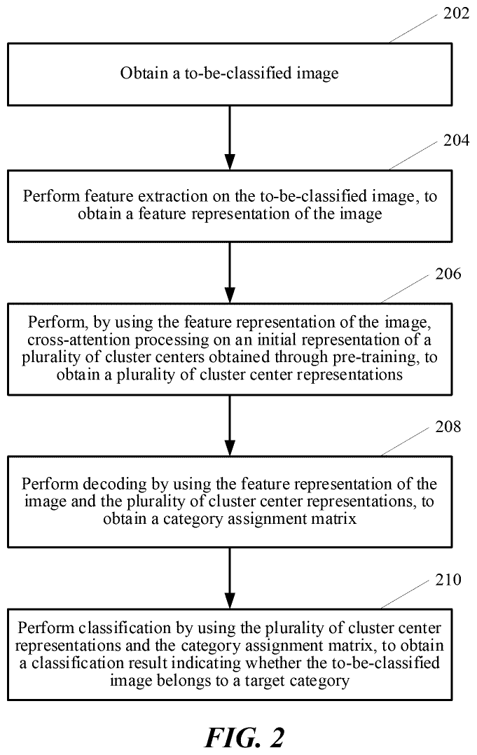

FIG. 2 is a flowchart of an image classification method according to an embodiment of this application. The method may be performed by the image classification apparatus in the system shown in FIG. 1. As shown in FIG. 2, the method may include the following steps.

Step 202: Obtain a to-be-classified image.

Step 204: Perform feature extraction on the to-be-classified image, to obtain a feature representation of the image.

Step 206: Perform, by using the feature representation of the image, cross-attention processing on an initial representation of a plurality of cluster centers obtained through pre-training, to obtain a plurality of cluster center representations.

Step 208: Perform decoding by using the feature representation of the image and the plurality of cluster center representations, to obtain a category assignment matrix.

Step 210: Perform classification by using the plurality of cluster center representations and the category assignment matrix, to obtain a classification result indicating whether the to-be-classified image belongs to a target category.

It can be seen from the foregoing procedure that, in this application, high-level semantics included in the plurality of cluster centers obtained through pre-training are jointly decoded with the feature representation of the image, to obtain the category assignment matrix, so that the category assignment matrix can reflect an image feature associated with the cluster center, e.g., the image feature is matched to a corresponding cluster center, thereby enabling an image classification result obtained based on the cluster center representation and the category assignment matrix to have a higher accuracy rate.

The above steps are described in detail below. First, in step 202, an example implementation of the action of obtaining a to-be-classified image can include the following details.

The to-be-classified image related in this application may be a two-dimensional image, or may be a three-dimensional image. The image may be a grayscale image, or may be a color image.

The to-be-classified image may include different content in different application fields. For example, in the field of transportation, the to-be-classified image is usually an image including a traffic element such as a vehicle, a pedestrian, a road, or a traffic facility. An objective of image classification may be to determine whether the image belongs to a specific traffic scene. For another example, in the field of e-commerce, the to-be-classified image is usually an image including commodity information, and an objective of image classification may be to determine whether the image belongs to a specific commodity category. For another example, in the field of medicine, the image is usually a medical image, for example, CT (computed tomography, computed tomography image), MRI (magnetic resonance imaging, magnetic resonance imaging image), or an ultrasound image, including organs such as a lung, a liver, a pancreas, and a colon. An objective of image classification may be to determine whether the image belongs to an organ area of a specific category, or whether there is an abnormality of a specific category.

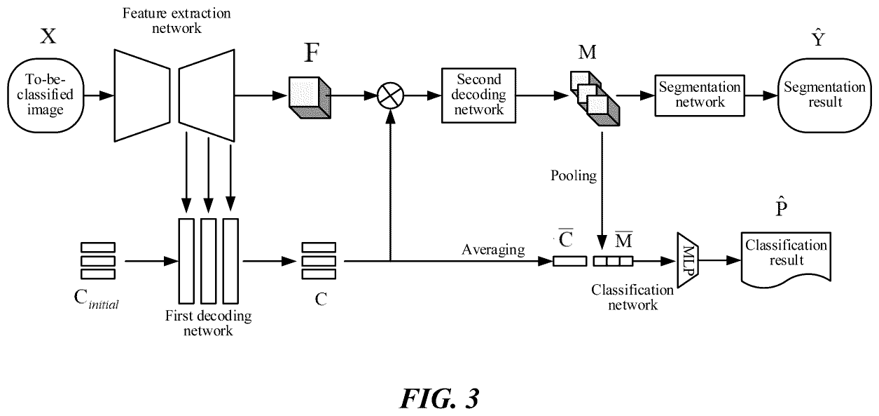

Steps 204 to 210 in the foregoing procedure may be implemented by using the image classification model obtained through pre-training. As shown in FIG. 3, the image classification model provided in embodiments of this application may include: a feature extraction network, a first decoding network, a second decoding network, and a classification network.

In step 204, the action of performing feature extraction on the to-be-classified image to obtain a feature representation of the image may be performed by the feature extraction network.

The feature extraction network may be implemented based on a transformer network, to extract an image feature, and obtain a feature representation of each Token (element) in the to-be-classified image. For example, the feature extraction network may use, for example, a VIT (vision transformer), a RAN (residual attention network), a U-Net (the U-Net is a variation of an FCN fully convolutional network), which is proposed to resolve a problem of a biomedical image, and is subsequently widely applied to various fields of image segmentation due to a very good effect), and the like.

Each Token of the image refers to an element constituting the image. For the image, the image is segmented into a non-overlapping block sequence, and a block and a sequence initiator in the image are both Tokens. For a two-dimensional image, a block in the image may include one or more pixels. For a three-dimensional image, a block in the image may include one or more voxels.

In an example implementation, for example, a U-Net may be used as the feature extraction network, to obtain a feature representation of each Token at a plurality of resolutions, thereby improving accuracy of image classification by using such a multi-scale feature. For example, the U-Net uses an Encoder (encoder)-Decoder (decoder) architecture. An Encoder performs downsampling, and a Decoder performs upsampling and feature concatenation, to obtain a plurality of feature representations at different resolutions, e.g., first obtain a feature representation of each Token at a low resolution, and then sequentially obtain a feature representation of each Token at a higher resolution. Then, a feature representation of each Token at a highest resolution is used as the feature representation of the image.

As shown in FIG. 3, it is assumed that an input to-be-classified image is represented as X, and a feature representation F of the image is obtained by using the feature extraction network, where F∈R^{A×HWD}. A is a feature dimension, and H, W, and D respectively represent a height, a width, and a length of a three-dimensional image (using an example in which the input to-be-classified image is the three-dimensional image).

In step 206, the action of performing, by using the feature representation of the image, cross-attention processing on an initial representation of a plurality of cluster centers obtained through pre-training, to obtain a plurality of cluster center representations may be performed by the first decoding network in the image classification model.

In this step, the first decoding network actually transforms an initial representation of a group of cluster centers into a cluster center representation fused with image semantics through cross attention of the feature representation of the image.

If the feature extraction network obtains the plurality of feature representations at different resolutions, in an example implementation, the first decoding network may include a multilayer concatenated Transformer network. A query matrix may be obtained by using the initial representation of the plurality of cluster centers, and the query matrix is input to a multilayer concatenated transformer network. Each layer of Transformer network is in one-to-one correspondence with each resolution based on an ascending order of resolutions. Each layer of Transformer network obtains a key matrix and a value matrix by using a feature representation of each Token at a corresponding resolution. Cross-attention processing is performed on the query matrix input to the layer of Transformer network, to obtain a query matrix output by the layer of Transformer network. The plurality of cluster center representations are obtained by using a query matrix output by a last layer of Transformer network.

Processing performed by each layer of Transformer network may be represented as follows:

$${C}_{n} = {C}_{n - 1} + arg {max}_{N} ( {Q ( K )}^{T} ) V , \qquad ( 1 )$$

where Cₙ represents a matrix corresponding to a plurality of cluster center representations output by an n^{th} layer of Transformer network, Cₙ₋₁ is a matrix corresponding to a plurality of cluster center representations output by an (n−1)^{th} layer of Transformer network, and for the first layer of Transformer network, and Cₙ₋₁ input by the first layer of Transformer network is a matrix Cᵢₙᵢₜᵢₐₗ corresponding to the initial representation of the plurality of cluster centers. Q is obtained from Cₙ₋₁, and K and V are both obtained through a feature representation of each Token at a resolution corresponding to the layer of Transformer network. T represents transpose processing. N is a quantity of cluster centers.

It can be seen from the foregoing process that, a first decoder is equivalent to grouping the Tokens in the image based on an initial cluster center. Cross-attention processing is similar to a K-means cluster algorithm, and argmax processing is used to replace Softmax processing in a transformer network. A finally obtained matrix corresponding to the plurality of cluster center representations is represented as C∈R^{N×A}.

In step 208, the action of performing decoding by using the feature representation of the image and the plurality of cluster center representations, to obtain a category assignment matrix may be performed by the second decoding network.

In this step, re-assignment is actually performed on the feature representations of the Tokens based on N cluster centers, and the feature representations are assigned to different clusters. Therefore, a result obtained in this step is referred to as the category assignment matrix.

The second decoding network may perform matrix multiplication on a matrix C corresponding to the plurality of cluster center representations and the feature representation F of the image, and then perform Softmax to obtain a matrix M corresponding to the category assignment matrix. The matrix M may be represented as:

$$M = {Softmax}_{N} ( CF ) , \qquad ( 2 )$$

where Softmax_{N}( ) represents processing of Softmax (which is a normalized exponential function) performed on an N-cluster center dimension.

In step 210, the performing classification by using the plurality of cluster center representations and the category assignment matrix, to obtain a classification result indicating whether the to-be-classified image belongs to a target category may be performed by the classification network in the image classification model shown in FIG. 3.

Because the learned plurality of cluster center representations have high-level semantics, and reflect a difference between the clusters and an intra-cluster similarity, the plurality of cluster center representations and the category assignment matrix are combined for classification, which can significantly improve accuracy and a recall rate of classification compared to directly using the feature representation of the image to perform classification.

In a possible implementation, averaging may be performed on the plurality of cluster center representations, to obtain a cluster-averaged representation; pooling may be performed on the category assignment matrix, to obtain a cluster-pooled representation; and then, the cluster-averaged representation and the cluster-pooled representation are integrated, and classification is performed by using a feature representation obtained through the integration, to obtain the classification result indicating whether the to-be-classified image belongs to the target category.

For example, the cluster-averaged representation may be obtained by performing averaging on the plurality of cluster center representations on a channel dimension. In addition to averaging, the matrix C may be transformed into a vector in another processing manner. In FIG. 3, the cluster-averaged representation is C̅.

When pooling is performed on the category assignment matrix, global maximum pooling, for example, may be used. In FIG. 3, the cluster-pooled representation obtained through pooling is M̅.

Integrating the cluster-averaged representation and the cluster-pooled representation may be achieved through concatenation. As shown in FIG. 3, after C̅ and M̅ are concatenated, the classification result \hat{P} indicating whether the to-be-classified image belongs to the target category is obtained through several layers of an MLP (multilayer perceptron).

In some embodiments, the classification result may be binary classification, or may be multi-class classification. Using the binary classification as an example, the output classification result may be that the to-be-classified image belongs to the target category, or that the to-be-classified image does not belong to the target category.

Further, in some embodiment, a segmentation network in the image classification model may alternatively segment the to-be-classified image by using the category assignment matrix, to obtain an image area of a preset category.

The category assignment matrix is obtained by re-assigning the feature representations of the Tokens based on the N cluster centers, to assign the feature representations to different clusters. Therefore, the Tokens belonging to a same cluster may be treated as a whole. Therefore, the category assignment matrix may be projected to K channels, thereby obtaining image areas of K categories, for example, a segmentation result Ŷ, where K is a preset positive integer greater than 1. The K categories include a target category corresponding to the classification network. The segmentation result of the to-be-classified image may provide interpretability for the classification result, for a user to refer to and understand an area of the target category in the to-be-classified image.

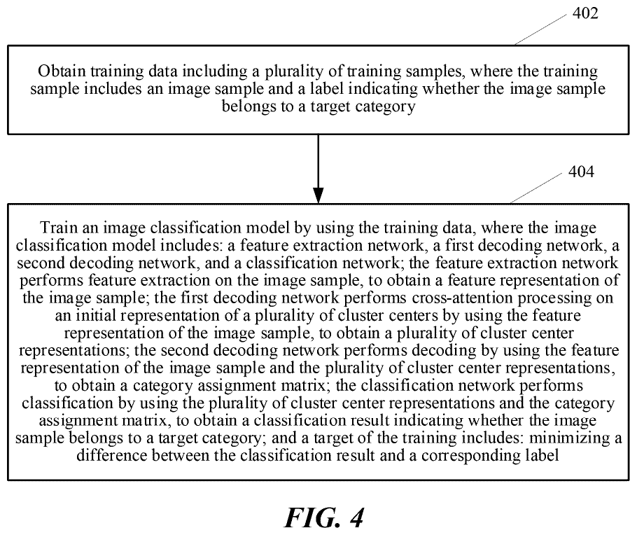

The following describes in detail a training process of the image classification model used in the foregoing embodiment. FIG. 4 is a flowchart of a method for training an image classification model according to an embodiment of this application. A procedure of the method may be performed by the model training apparatus in the system shown in FIG. 1. As shown in FIG. 4, the method may include the following steps.

Step 402: Obtain training data including a plurality of training samples, where the training sample includes an image sample and a label indicating whether the image sample belongs to a target category.

In some embodiments, some images that are known to belong to the target category or not belong to the target category may be obtained as image samples, and the label indicating whether the image sample belongs to the target category is marked on the image sample. Alternatively or additionally, some images are obtained as image samples, and the label indicating whether the image sample belongs to the target category is manually marked on the image sample. These image samples may be obtained from a specific application field based on an actual requirement.

In some implementations, the image sample may further be marked with an area mask (Mask) of a preset category, e.g., an area of the preset category in the image sample is marked, to form an area mask of a specific category. A manual marking manner may be used, or an image sample of the known area of the preset category may be obtained. An area of the preset category in the image sample is marked, to form an area mask. The preset category includes the target category.

For example, a training data set S may be represented as: {(Xᵢ, Yᵢ, Pᵢ)|i=1, 2, . . . , m}, where Xᵢ is an image sample, Pᵢ may be represented as a label indicating whether Xᵢ belongs to a target category marked on Xᵢ, Yᵢ is an area mask of a preset category marked on Xᵢ, and m is a quantity of training samples.

Step 404: Train an image classification model by using the training data, where the image classification model includes: a feature extraction network, a first decoding network, a second decoding network, and a classification network. The feature extraction network performs feature extraction on the image sample, to obtain a feature representation of the image sample. The first decoding network performs cross-attention processing on an initial representation of a plurality of cluster centers by using the feature representation of the image sample, to obtain a plurality of cluster center representations. The second decoding network performs decoding by using the feature representation of the image sample and the plurality of cluster center representations, to obtain a category assignment matrix. The classification network performs classification by using the plurality of cluster center representations and the category assignment matrix, to obtain a classification result indicating whether the image sample belongs to the target category. A target of the training includes: minimizing a difference between the classification result and a corresponding label.

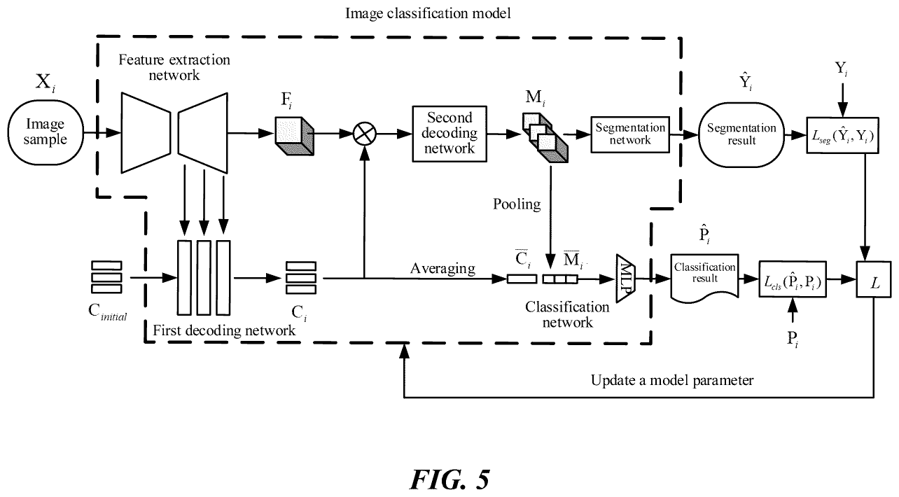

As shown in FIG. 5, the feature extraction network performs feature extraction on the image sample Xᵢ, to obtain a feature representation Fⱼ of the image sample. The feature extraction network may be implemented based on a transformer network, to extract an image feature, and obtain a feature representation of each Token (element) in the image sample.

In a possible implementation, the feature extraction network may perform feature extraction on the image sample, to sequentially obtain a feature representation of each element Token at a plurality of resolutions, and determine a feature representation of each Token at a highest resolution as the feature representation of the image sample. For example, a U-Net may be used as the feature extraction network, to obtain the feature representation of each Token at the plurality of resolutions, and accuracy of image classification is improved by using such a multi-scale feature. For example, the U-Net uses an encoder-decoder architecture. An encoder performs downsampling, and a decoder performs upsampling and feature concatenation, to obtain a plurality of feature representations at different resolutions. For example, some implementations first obtain a feature representation of each token at a low resolution, and then sequentially obtain a feature representation of each token at a higher resolution. Then, a feature representation of each Token at a highest resolution is used as the feature representation of the image.

The first decoding network performs cross-attention processing on the initial representation (a corresponding matrix is represented as Cᵢₙᵢₜᵢₐₗ) of the plurality of cluster centers by using the feature representation Fᵢ of the image sample, to obtain the plurality cluster center representations (a corresponding matrix is represented as Cᵢ).

If the feature extraction network obtains the plurality of feature representations at different resolutions, in an example implementation, the first decoding network may include a multilayer concatenated transformer network, the initial representation of the plurality of cluster centers is determined as a query matrix input to a first layer of transformer network, each layer of transformer network is in one-to-one correspondence with each resolution based on an ascending order of resolutions, each layer of transformer network obtains a key matrix and a value matrix by using a feature representation of each token at a corresponding resolution, and cross-attention processing is performed on the query matrix input to the layer of Transformer network, to obtain a query matrix output by the layer of transformer network; and the plurality of cluster center representations are obtained by using a query matrix output by a last layer of transformer network.

The second decoding network performs decoding by using the feature representation Fᵢ of the image sample and the plurality of cluster center representations Cᵢ, to obtain a category assignment matrix Mᵢ. Re-assignment is actually performed on the feature representations of the Tokens based on the plurality of cluster centers, and the feature representations are assigned to different clusters. Therefore, a result obtained in the second decoding network is referred to as the category assignment matrix.

The classification network performs classification by using the plurality of cluster center representations Cᵢ and the category assignment matrix Mᵢ, to obtain a classification result indicating whether the image sample belongs to the target category.

In a possible implementation, the classification network may perform averaging on the plurality of cluster center representations, to obtain a cluster-averaged representation; the classification network may perform pooling on the category assignment matrix, to obtain a cluster-pooled representation; and the classification network may integrate the cluster-averaged representation and the cluster-pooled representation, and perform classification by using a feature representation obtained through integration, to obtain the classification result indicating whether the image sample belongs to the target category.

For example, the cluster-averaged representation may be obtained by performing averaging on the plurality of cluster center representations on a channel dimension. In addition to averaging, the matrix Cᵢ may be transformed into a vector in another processing manner. In FIG. 5, the cluster-averaged representation is C̅ᵢ.

When pooling is performed on the category assignment matrix, global maximum pooling, for example, may be used. In FIG. 5, the cluster-pooled representation obtained through pooling is M̅ᵢ.

Integrating the cluster-averaged representation and the cluster-pooled representation may be performing concatenation. As shown in FIG. 5, after C̅ᵢ and M̅ᵢ are concatenated, a classification result \hat{P}ᵢ indicating whether a to-be-classified image belongs to the target category is obtained through several layers of an MLP (Multilayer Perceptron, multilayer perceptron).

In embodiments of this application, the classification result may be binary classification, or may be multi-class classification. Using the binary classification as an example, the output classification result may be that the to-be-classified image belongs to the target category, or that the to-be-classified image does not belong to the target category.

During training, a target of the training includes minimizing a difference between the classification result \hat{P}ᵢ and a label Pᵢ marked for Xᵢ in the training sample.

Still further, in some embodiments, a segmentation network in the image classification model may further segment the image sample by using the category assignment matrix, to obtain an image area of a preset category.

The category assignment matrix is obtained by re-assigning the feature representations of the Tokens based on the plurality of cluster centers, to assign the feature representations to different clusters. Therefore, the Tokens belonging to a same cluster may be treated as a whole. Therefore, the category assignment matrix may be projected to K channels, thereby obtaining image areas of K categories, that is, a segmentation result Ŷᵢ, where K is a preset positive integer greater than 1. The K categories include a target category corresponding to the classification network.

During training, the target of the training may further include minimizing a difference between the image area Ŷᵢ of the preset category and a corresponding area mask Yᵢ. The target of the training may be used to assist learning of the classification result in the model training process.

In a possible implementation, a loss function may be constructed based on the target of the training. A model parameter is updated in each iteration by using a value of the loss function in a manner such as gradient descent, until a preset training end condition is satisfied. The training end condition may include, for example, that the value of the loss function is less than or equal to a preset loss function threshold, or that a quantity of iterations reaches a preset quantity threshold.

If the foregoing two targets of the training are used, a total loss function L may be constructed. For example:

$$L = \frac{1}{b} {\sum }_{i = 1}^{b} {L}_{seg} ( {\hat{Y}}_{i} , {Y}_{i} ) + {L}_{cls} ( {\hat{P}}_{i} , {P}_{i} ) , \qquad ( 3 )$$

where b is a quantity of training samples in a batch (batch), L_{seg}(Ŷᵢ, Yᵢ) reflects a difference between Ŷᵢ and Yᵢ, and L_{cls}(\hat{P}ᵢ, Pᵢ) reflects a difference between \hat{P}ᵢ and Pᵢ.

The image classification method provided in some embodiments may be applied to a plurality of scenarios. For example, a remote sensing image may be used as the to-be-classified image to perform mineral crystal detection, oil exploration, or the like. For example, the remote sensing image is classified by using the method provided in some embodiments, to determine whether the remote sensing image belongs to a target mineral crystal category. Meanwhile, the remote sensing image may be segmented to obtain areas of various categories, including a mineral crystal area, thereby providing interpretable reference. For example, the method may be applied to device abnormality detection in some severe environments. For example, a device such as an unmanned aerial vehicle or an inspection robot may be used to acquire a device image, the device image is used as the to-be-classified image, and the device image is classified by using the method provided in some embodiment of this specification, to determine whether the device image belongs to an abnormality category, e.g., whether an abnormality exists. In addition, the device image may be segmented to obtain a background area, a device area, an abnormal area, and the like, to provide interpretable reference.

The methods provided in some embodiments of this specification may be further applied to other use scenarios, for example, medical scenario. A medical image may be classified by using the method provided in embodiments of this application, to determine whether a specific focus exists. The application scenario is described below by using an example.

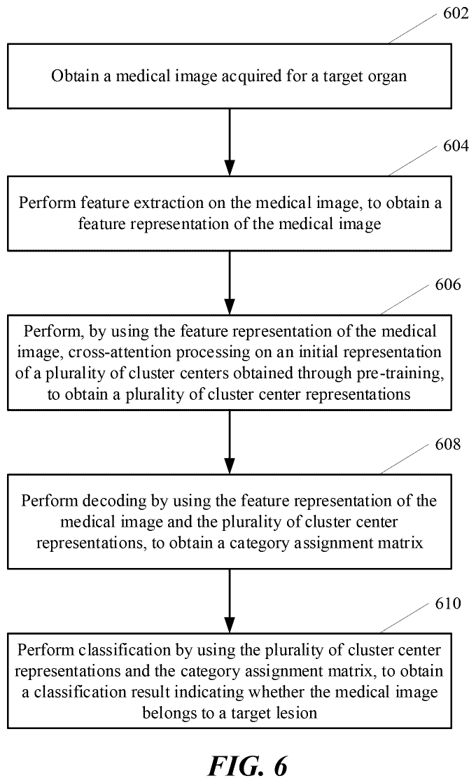

FIG. 6 is a flowchart of an image classification method applied to the medical scenario according to some embodiment of this specification. As shown in FIG. 6, the method may, in some implementations, include the following steps.

Step 602: Obtain a medical image acquired for a target organ.

The acquired medical image herein uses a non-invasive acquisition manner, such as CT (computed tomography), MRI (magnetic resonance imaging), and an ultrasound scan. The target organ may be an organ such as a stomach, a lung, a liver, a pancreas, or a colon. For example, the medical image may be a non-contrast CT image including a stomach, or the like.

Using the non-contrast CT image including a stomach as an example, usually, a multilayer image is generated when a non-contrast CT device scans one circle. Therefore, the non-contrast CT image X∈R^{H×W×D}, where H, W, and D respectively represent a height, a width, and a quantity of layers of a three-dimensional image (using an example in which an input to-be-classified image is the three-dimensional image).

Step 604: Perform feature extraction on the medical image, to obtain a feature representation of the medical image.

Step 606: Perform, by using the feature representation of the medical image, cross-attention processing on an initial representation of a plurality of cluster centers obtained through pre-training, to obtain a plurality of cluster center representations.

For steps 604 and 606, refer to the records for steps 204 and 206 in the foregoing embodiments. Details are not described herein.

A quantity N of cluster centers is a hyperparameter. A value of N is usually greater than a quantity of categories K of subsequent segmented areas. Because lesions such as a stomach cancer may have a plurality of sub-species, for example, features reflected by different development stages of the stomach cancer are different, a larger quantity of cluster centers may be selected. N is generally taken as an empirical value or an experimental value. For example, the value is taken as 8.

Step 608: Perform decoding by using the feature representation of the medical image and the plurality of cluster center representations, to obtain a category assignment matrix.

Step 610: Perform classification by using the plurality of cluster center representations and the category assignment matrix, to obtain a classification result indicating whether the medical image belongs to a target lesion.

For steps 608 and 610, refer to the descriptions of steps 208 and 210 in the foregoing embodiments for example details. Further details are not described herein for brevity purposes.

Using the non-contrast CT image of the stomach as an example, the classification result obtained in step 610 may be a binary classification result, for example, being normal or having the target stomach cancer. In this way, a result of detecting whether there is a suspicion of having the stomach cancer based on the non-contrast CT image of the stomach is implemented, to be provided to a doctor for reminding and reference. Cross-attention processing is performed on the initial feature representation of the plurality of cluster centers based on a feature representation of each Token at a plurality of resolutions, and the feature representation of the plurality of cluster centers and the feature representation of the image are re-assigned, so that a classification process can be sensitive to a local tissue structure, and has global awareness of organ physiology.

Still further, in embodiments of this application, the medical image may further be segmented by a segmentation network in an image classification model by using the category assignment matrix, to obtain an image area of a preset category. The preset category may include a background, an organ, and a focus. Still using the non-contrast CT image of the stomach as an example, a background area, a stomach area, and a stomach cancer area may be obtained through segmentation, to be provided to the doctor as interpretable reference.

In embodiments of this application, feature extraction may further be performed on the medical image, to obtain a feature representation of each element Token at a plurality of resolutions, and a feature representation of each Token at a highest resolution is determined as the feature representation of the medical image.

If a feature extraction network obtains a plurality of feature representations at different resolutions, in an example implementation, a query matrix may be obtained by using the initial representation of the plurality of cluster centers, the query matrix is input to a multilayer concatenated transformer network, each layer of transformer network is in one-to-one correspondence with each resolution based on an ascending order of resolutions, each layer of transformer network obtains a key matrix and a value matrix by using a feature representation of each Token at a corresponding resolution, and cross-attention processing is performed on the query matrix input to the layer of transformer network, to obtain a query matrix output by the layer of transformer network; and the plurality of cluster center representations are obtained by using a query matrix output by a last layer of Transformer network.

In a possible implementation, averaging may be performed on the plurality of cluster center representations, to obtain a cluster-averaged representation; pooling may be performed on the category assignment matrix, to obtain a cluster-pooled representation; and then, the cluster-averaged representation and the cluster-pooled representation are integrated, and classification is performed by using a feature representation obtained through integration, to obtain the classification result indicating whether the medical image belongs to the target lesion. In embodiments of this application, a computer-aided diagnosis method for a stomach cancer is further provided. The method includes:
obtaining a medical image acquired for a stomach;
performing feature extraction on the medical image, to obtain a feature representation of the medical image;
performing, by using the feature representation of the medical image, cross-attention processing on an initial representation of a plurality of cluster centers obtained through pre-training, to obtain a plurality of cluster center representations;
performing decoding by using the feature representation of the medical image and the plurality of cluster center representations, to obtain a category assignment matrix; and
performing classification by using the plurality of cluster center representations and the category assignment matrix, to obtain a classification result indicating whether the medical image belongs to a stomach cancer.

When the procedure shown in FIG. 4 is used to train an image classification model for detecting the stomach cancer, an image sample may be a non-contrast CT image of a stomach known as being normal or having the stomach cancer, which is marked as Xᵢ. Each non-contrast CT image is marked with a label Pᵢ of being normal or having the stomach cancer. Because it is difficult to mark a non-contrast CT area, after a doctor marks a background area, a stomach area, and a stomach cancer area in a contrast CT (Contrast CT) image corresponding to each Xᵢ, the marked contrast CT image is aligned with the non-contrast CT image, to form relatively rough but highly reliable area masks.

The stomach cancer is a third leading cause of cancer-related deaths worldwide, and a 5-year survival rate is approximately 33%. If a related symptom can be detected at an early stage, the 5-year survival rate can be significantly improved. Because an early stomach tumor may invade only a mucous membrane and muscularis, it is very difficult to identify the stomach tumor if a stomach contrast agent is not injected. Currently, existing detection manners such as barium meal gastrography, endoscopy, and a serum pepsinogen level test are invasive, costly, and associated with significant side effects, and are difficult to be well applied to the early detection of stomach cancer. By using the foregoing manner provided in embodiments of this application, an image can be obtained in a non-invasive and low-cost manner such as the non-contrast CT image of the stomach. In addition, by using the manner provided in embodiments of this application, a computer device performs image classification to detect the stomach cancer. In other words, a finally output classification result is whether the image belongs to the stomach cancer. For example, a binary classification result of being normal or having the stomach cancer is output, to be used as intermediate data to provide reference basis or a reminder for the doctor or a patient, to facilitate further examination and diagnosis subsequently. In addition, image segmentation can be performed on the non-contrast CT image of the stomach, to obtain a background area, a stomach area, and a focal area (e.g., an area in which the stomach cancer is located) through segmentation, to be used as intermediate data to provide interpretable reference for the doctor or the patient. It is clear that this is a new detection method that is non-invasive, low-cost, and easy to be promoted, and the method has a good effect in terms of precision.

Experiments are performed. According to the method provided in embodiments of this application, on a test set of non-contrast CT images of a stomach from 100 stomach cancer patients and 148 normal patients, a sensitivity of 85.0% and a specificity of 92.6% are achieved in obtaining a classification result of having the stomach cancer through classification. However, an average sensitivity of manual stomach cancer detection by a radiologist on the non-contrast CT images of the stomach is 73.5%, with a specificity of 84.3%. It is clear that an image classification method based on a computer vision technology provided in embodiments of this application has a better effect. The sensitivity is also referred to as a true positive rate, and refers to a probability that a classification result is having the stomach cancer when a stomach cancer sample is tested. The specificity is also referred to as a true negative rate, and refers to a probability that a classification result is being normal when a normal sample is tested.

Example embodiments of this specification are described above. Other embodiments are within the scope of the following claims. In some cases, the actions or steps recorded in the claims may be performed in sequences different from those in the embodiments and an expected result may still be achieved. In addition, the processes described in the accompanying drawings do not necessarily require that an expected result can only be achieved in a shown specific order or sequential order. In some implementations, multitasking processing and parallel processing may alternatively be possible, or may be advantageous.

Refer to the following Table 1. Table 1 outputs a comparison analysis between the image classification method according to the present disclosure and three benchmarks. Two methods used by the first two benchmarks belong to “classification and segmentation” (S4C), and use nnUNet and TransUNet. † represents that p of a DeLong test (the present disclosure and nnUNet-S4C) is lower than 0.05; and * represents that p of a permutation test (the present disclosure, nnUNet-S4C, and the radiographer) is lower than 0.05. A case is classified as positive if a segmented tumor volume exceeds a threshold that maximizes a sum of a sensitivity and a specificity of a validation set. A third baseline (which is represented as “nnUNet-Joint”) integrates a CNN classification head into UNet, and performs end-to-end training. A 95% confidence interval of values of an AUC, a sensitivity, and a specificity may be obtained from 1000 bootstrap copies of a test data set, for statistical analysis. To obtain statistical significance, the DeLong test may be performed between two AUCs (the image classification method according to the present disclosure and a comparison method), and the permutation test may be performed between two sensibilities or specificities (the image classification method according to the present disclosure, the comparison method, and the radiologist).

TABLE 1

| | Internal hold-out (internal hold-out) (n = 248) | | | External (n = 903) |
|---|---|---|---|---|
| | | Sensitivity | Specificity | Specificity |
| Method | AUC | (%) | (%) | (%) |
| Radiologist | — | 73.5 | 84.1 | — |
| nnUNet-S4C | 0.907 | 80 | 88.5 | 96.6 |
| | (0.862, 0.942) | (72.0, 87.5) | (83.3, 93.5) | (95.2, 97.8) |
| TransUNet-S4C | 0.916 | 82 | 90.5 | 96 |
| | (0.876, 0.952) | (74.7, 89.5) | (86.1, 94.8) | (94.8, 97.2) |
| nnUNet-Joint | 0.924 | 81 | 90.5 | 97.6 |
| | (0.885, 0.959) | (73.0, 87.9) | (85.1, 95.0) | (96.5, 98.6) |
| Image classification | 0.939† | 85.0* | 92.6* | 97.7 |
| method of the | (0.910, 0.964) | (78.1, 91.1) | (88.0, 96.5) | (96.7, 98.7) |
| present disclosure | | | | |

Refer to Table 1. The image classification method of the present disclosure is better than the other three benchmarks (Table 1) in terms of all indexes, especially in terms of the AUC and the sensitivity. An advantage of the method according to the present disclosure is that the method captures both local information and global information by using a unique architecture of a mask Transformer. The method further extracts high-level semantics from a cluster representation, making it suitable for classification and facilitating an overall decision process. In addition, the method according to the present disclosure reaches a quite large specificity of 97.7% in an external test set, which is crucial for reducing false positives and unnecessary manpower workload in terms of opportunistic screening.

An ROC curve of the model provided in the present disclosure is better than a result obtained by performing detection by two experienced radiologists. The model achieves the sensitivity of 85.0% in detecting the stomach cancer, significantly exceeds an average performance of the doctor (73.5%), and also exceeds a best performance of the doctor (75.0%), while maintaining a high specificity.

Refer to Table 2. Performances of patient-level detection and tumor-level positioning are layered based on a tumor (T) stage. Table 2 compares performance of the model according to the present disclosure with performance of the two radiologists. A result indicates that the model according to the present disclosure performs better in detecting an early-stage tumor (T1 and T2) and provides more precise tumor positioning. For example, the model according to the present disclosure detects 60.0% (6/10) T1 cancers and 77.8% (7/9) T2 cancers, which exceeds a best performance of an expert (50% T1, 55.6% T2). In addition, in the model according to the present disclosure, a reliable detection rate and a credible positioning precision are maintained for T3 and T4 tumors (2 of 34 T3 tumors are missed for detection).

TABLE 2
Patient-level detection and tumor-level positioning
results (%) of different T-stage stomach cancers

| | | | | | | T-stage |
|---|---|---|---|---|---|---|
| | | | | | | information |
| Method | Standard | T1 | T2 | T3 | T4 | not available |
| nnUNet-S4C | Patient | 30.0 (3/10) | 66.7 (6/9) | 94.1 (32/34) | 100.0 (9/9) | 86.1 (31/36) |
| | Tumor | 20.0 (2/10) | 55.6 (5/9) | 94.1 (32/34) | 100.0 (9/9) | 80.6 (29/36) |
| The present | Patient | 60.0 (6/10) | 77.8 (7/9) | 94.1 (32/34) | 100.0 (9/9) | 86.1 (31/36) |
| disclosure | Tumor | 30.0 (3/10) | 66.7 (6/9) | 94.1 (32/34) | 100.0 (9/9) | 80.6 (30/36) |
| Radiologist 1 | Patient | 50.0 (5/10) | 55.6 (5/9) | 76.5 (26/34) | 88.9 (8/9) | 77.8 (28/36) |
| Radiologist 2 | Patient | 30.0 (3/10) | 55.6 (5/9) | 85.3 (29/34) | 100.0 (9/9) | 80.6 (29/36) |

As shown in Table 3, when a relatively large test patient volume (n=1151 by integrating an internal test set and the external test set), the method according to the present disclosure goes beyond or is equivalent to an existing screening tool in terms of sensitivity of stomach cancer detection, and has a similar specificity level, as shown in Table 3. * represents that two in-situ tumors are removed in the test set. † represents that only early stomach cancer cases are taken into consideration in the comparison, including the in-situ tumor, the T1-stage tumor, and the T2-stage tumor, where the method according to the present disclosure successfully detects 17 cases out of 19 cases.

TABLE 3
Comparison of performance of state-of-the-art blood detection
applied to stomach cancer detection, UGIS, and endoscopy screening
in large-scale population, and early stomach cancer detection
rates of senior radiologists on magnifying endoscopy with
narrow-band imaging (magnifying endoscopy, ME-NBI)

| | | | Sensitivity |
|---|---|---|---|
| | | | (%) of the |
| | | | present |
| | | | disclosure |
| | Specification | Sensitivity | with a same |
| Method | (%) | (%) | specificity |
| Blood detection | 99.5 | 66.7 | 69.4* |
| Upper gastrointestinal | 96.1 | 36.7 | 85.0 |
| series | | | |
| Endoscopy examination | 96.0 | 69.0 | 85.0 |
| ME-NBI (early-stage) | 74.2 | 76.7 | 89.5† |

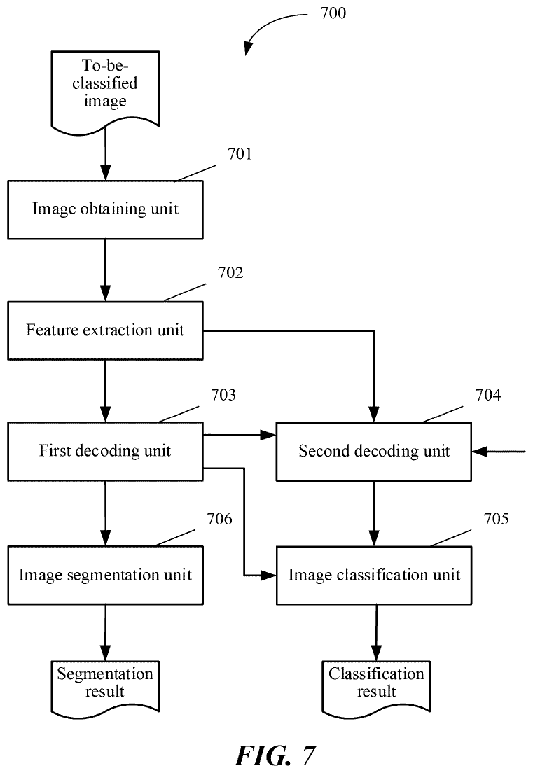

According to an embodiment in another aspect, an image classification apparatus is provided. FIG. 7 is a schematic block diagram of an image classification apparatus according to an embodiment. As shown in FIG. 7, the apparatus 700 includes an image obtaining unit 701, a feature extraction unit 702, a first decoding unit 703, a second decoding unit 704, and an image classification unit 705; and may further include an image segmentation unit 706. Main functions of the constituent units are as follows.

The image obtaining unit 701 is configured to obtain a to-be-classified image.

The feature extraction unit 702 is configured to perform feature extraction on the to-be-classified image, to obtain a feature representation of the image.

The first decoding unit 703 is configured to perform, by using the feature representation of the image, cross-attention processing on an initial representation of a plurality of cluster centers obtained through pre-training, to obtain a plurality of cluster center representations.

The second decoding unit 704 is configured to perform decoding by using the feature representation of the image and the plurality of cluster center representations, to obtain a category assignment matrix.

The image classification unit 705 is configured to perform classification by using the plurality of cluster center representations and the category assignment matrix, to obtain a classification result indicating whether the to-be-classified image belongs to a target category.

Still further, the image classification unit 705 is configured to perform segmentation on the to-be-classified image by using the category assignment matrix, to obtain an image area of a preset category. The preset category includes the target category.

In a possible implementation, the feature extraction unit 702 may be, for example, configured to: perform feature extraction on the to-be-classified image, to obtain a feature representation of each element Token at a plurality of resolutions, and determine a feature representation of each Token at a highest resolution as the feature representation of the image.

Correspondingly, the first decoding unit 703 may obtain a query matrix by using the initial representation of the plurality of cluster centers, and the query matrix is input to a multilayer concatenated transformer network. Each layer of Transformer network is in one-to-one correspondence with each resolution based on an ascending order of resolutions. Each layer of Transformer network obtains a key matrix and a value matrix by using a feature representation of each Token at a corresponding resolution. Cross-attention processing is performed on the query matrix input to the layer of Transformer network, to obtain a query matrix output by the layer of Transformer network. The plurality of cluster center representations are obtained by using a query matrix output by a last layer of Transformer network.

In a possible implementation, the image classification unit 705 may be, for example configured to: perform averaging on the plurality of cluster center representations, to obtain a cluster-averaged representation; perform pooling on the category assignment matrix, to obtain a cluster-pooled feature; and integrate the cluster-averaged representation and the cluster-pooled representation, and perform classification by using a feature representation obtained through integration, to obtain the classification result indicating whether the to-be-classified image belongs to the target category.

The feature extraction unit 702, the first decoding unit 703, the second decoding unit 704, the image classification unit 705, and the image segmentation unit 706 are respectively corresponding to the feature extraction network, the first decoding network, the second decoding network, the classification network, and the segmentation network in the image classification model shown in FIG. 3. For details, refer to the related records for FIG. 3 in the foregoing method embodiments, and details are not described herein again.

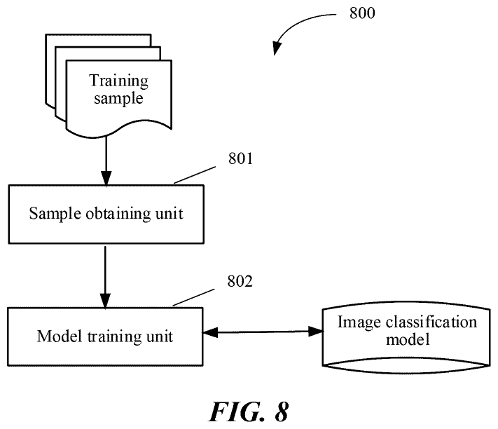

FIG. 8 is a schematic block diagram of an apparatus for training an image classification model according to an embodiment of this application. As shown in FIG. 8, the apparatus may include a sample obtaining unit 801 and a model training unit 802. Main functions of the constituent units are as follows.

The sample obtaining unit 801 is configured to obtain training data including a plurality of training samples, where the training sample includes an image sample and a label indicating whether the image sample belongs to a target category.

The model training unit 802 is configured to train an image classification model by using the training data, where the image classification model includes: a feature extraction network, a first decoding network, a second decoding network, and a classification network. The feature extraction network performs feature extraction on the image sample, to obtain a feature representation of the image sample. The first decoding network performs cross-attention processing on an initial representation of a plurality of cluster centers by using the feature representation of the image sample, to obtain a plurality of cluster center representations. The second decoding network performs decoding by using the feature representation of the image sample and the plurality of cluster center representations, to obtain a category assignment matrix. The classification network performs classification by using the plurality of cluster center representations and the category assignment matrix, to obtain a classification result indicating whether the image sample belongs to the target category. A target of the training includes: minimizing a difference between the classification result and a corresponding label.

In a possible implementation, the training sample may further include an area mask of a preset category marked on the image sample. The image classification model further includes a segmentation network.

The segmentation network segments the image sample by using the category assignment matrix, to obtain an image area of the preset category, where the preset category includes the target category. Correspondingly, the target of the training may further include minimizing a difference between the image area of the preset category and a corresponding area mask.

In a possible implementation, the feature extraction network may perform feature extraction on the image sample, to sequentially obtain a feature representation of each element Token at a plurality of resolutions, and determine a feature representation of each Token at a highest resolution as the feature representation of the image sample.

Correspondingly, the first decoding network may include a multilayer concatenated Transformer network, the initial representation of the plurality of cluster centers is determined as a query matrix input to a first layer of Transformer network, each layer of Transformer network is in one-to-one correspondence with each resolution based on an ascending order of resolutions, each layer of Transformer network obtains a key matrix and a value matrix by using a feature representation of each Token at a corresponding resolution, and cross-attention processing is performed on the query matrix input to the layer of Transformer network, to obtain a query matrix output by the layer of Transformer network; and the plurality of cluster center representations are obtained by using a query matrix output by a last layer of Transformer network.

In a possible implementation, the classification network may perform averaging on the plurality of cluster center representations, to obtain a cluster-averaged representation; the classification network may perform pooling on the category assignment matrix, to obtain a cluster-pooled representation; and the classification network may integrate the cluster-averaged representation and the cluster-pooled representation, and perform classification by using a feature representation obtained through integration, to obtain the classification result indicating whether the image sample belongs to the target category.

According to an embodiment in another aspect, an image classification apparatus is provided, applied to medical diagnosis. The apparatus includes: an image obtaining unit, a feature extraction unit, a first decoding unit, a second decoding unit, and an image classification unit, and may further include an image segmentation unit. Main functions of the constituent units are as follows.

The image obtaining unit is configured to obtain a medical image acquired for a target organ.

The feature extraction unit is configured to perform feature extraction on the medical image, to obtain a feature representation of the medical image.

The first decoding unit is configured to perform, by using the feature representation of the medical image, cross-attention processing on an initial representation of a plurality of cluster centers obtained through pre-training, to obtain a plurality of cluster center representations.

The second decoding unit is configured to perform decoding by using the feature representation of the medical image and the plurality of cluster center representations, to obtain a category assignment matrix.

The image classification unit is configured to perform classification by using the plurality of cluster center representations and the category assignment matrix, to obtain a classification result indicating whether the medical image belongs to a target lesion.

Various embodiments of this specification are all described in a progressive manner. For the same or similar parts between the various embodiments, refer to these embodiments. Each embodiment focuses on a difference from another embodiment. Especially, apparatus embodiments are basically similar to the method embodiments, and therefore are described briefly. For related parts, refer to partial descriptions in the method embodiments. The foregoing described apparatus embodiments are merely examples. The units described as separate parts may or may not be physically separate, and the parts displayed as units may or may not be physical units, may be located in one position, or may be distributed on a plurality of network units. Some or all of the modules may be selected based on an actual requirement to implement the objectives of the solutions of the embodiments. A person of ordinary skill in the art may understand and implement the solutions of the embodiments without creative efforts.

It should be noted that, user information (including but not limited to user device information, user personal information, and the like) and data (including but not limited to data for analysis, data for storage, data for display, and the like) involved in this application are all information and data authorized by a user or fully authorized by each party, and collection, use, and processing of related data need to comply with relevant laws, regulations and standards of related countries and regions. In addition, a corresponding operation entry is provided for the user to choose authorization or rejection.

In addition, an embodiment of this application further provides a computer-aided diagnosis system for a cancer, including a memory, a processor, and a computer program stored in the memory and run on the processor, where the processor, when executing the computer program, is operable to perform an image classification method, and the method includes:
obtaining a medical image acquired for a target organ;
performing feature extraction on the medical image, to obtain a feature representation of the medical image;
performing, by using the feature representation of the medical image, cross-attention processing on an initial representation of a plurality of cluster centers obtained through pre-training, to obtain a plurality of cluster center representations;
performing decoding by using the feature representation of the medical image and the plurality of cluster center representations, to obtain a category assignment matrix; and performing classification by using the plurality of cluster center representations and the category assignment matrix, to obtain a classification result indicating whether the medical image belongs to a target lesion.

In addition, an embodiment of this application further provides a computer-readable storage medium. The medium stores a computer program. The program, when executed by a processor, implements the steps of the method according to any one of the foregoing method embodiments.

Moreover, an electronic device is provided, including:
one or more processors; and
a memory associated with the one or more processors, where the memory is configured to store program instructions, and the program instructions, when read and executed by the one or more processors, implement steps of the method according to any one of the foregoing method embodiments.

This application further provides a computer program product, including a computer program. The computer program, when executed by a processor, implements the steps of the method according to any one of the foregoing method embodiments.

This application further provides a computer program. The computer program, when executed by a computer, causes the computer to perform steps of the method according to any one of the foregoing method embodiments.

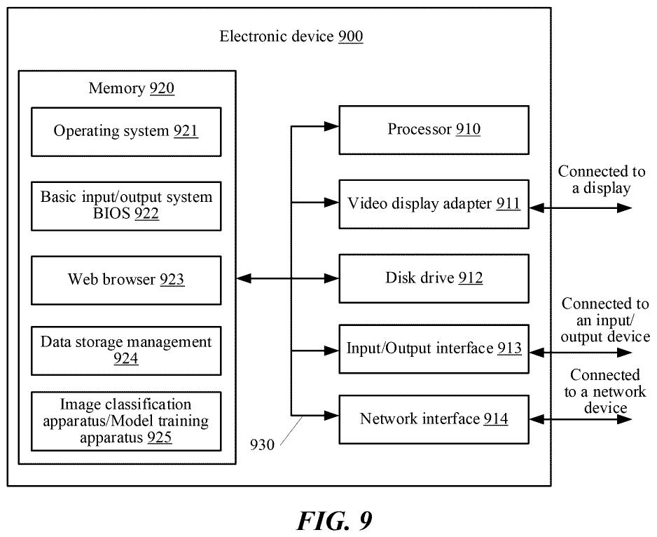

FIG. 9 illustratively shows an architecture of an electronic device, which may, in some implementations, include a processor 910, a video display adapter 911, a disk drive 912, an input/output interface 913, a network interface 914, and a memory 920. The processor 910, the video display adapter 911, the disk drive 912, the input/output interface 913, the network interface 914, and the memory 920 may be communicatively connected by using a communication bus 930.

The processor 910 may be implemented in a manner such as a general-purpose CPU, a microprocessor, an application specific integrated circuit (application specific integrated circuit, ASIC), or one or more integrated circuits, and is configured to perform a related program, to implement the technical solutions provided in this application.

The memory 920 may be implemented in a form such as a ROM (Read Only Memory, read only memory), a RAM (Random Access Memory, random access memory), a static storage device, and a dynamic storage device. The memory 920 may store an operating system 921 configured to control running of the electronic device 900, and a basic input/output system (BIOS) 922 configured to control a low-level operation of the electronic device 900. In addition, the memory 920 may further store a web browser 923, a data storage management system 924, an image classification apparatus/model training apparatus 925, and the like. The image classification apparatus/model training apparatus 925 may be an application program that implements the operations of the foregoing steps in the embodiments of this application. In conclusion, when the technical solutions provided in this application are implemented by using software or firmware, related program code is stored in the memory 920, and is called and executed by the processor 910.

The input/output interface 913 is configured to be connected to an input/output module, to implement information input and output. The input/output module may be configured in the device as a component (not shown in the figure), or may be externally connected to the device to provide a corresponding function. An input device may include a keyboard, a mouse, a touchscreen, a microphone, various sensors, and the like. An output device may include a display, a speaker, a vibrator, an indicator, and the like.

The network interface 914 is configured to be connected to a communication module (not shown in the figure), to implement communication and interaction between the device and another device. The communication module may implement communication in a wired manner (such as a USB or a network cable), or may implement communication in a wireless manner (such as a mobile network, WIFI, or Bluetooth).

The bus 930 includes a path for transmitting information between the components (such as the processor 910, the video display adapter 911, the disk drive 912, the input/output interface 913, the network interface 914, and the memory 920) of the device.

It should be noted that, although the foregoing device only shows the processor 910, the video display adapter 911, the disk drive 912, the input/output interface 913, the network interface 914, the memory 920, the bus 930, and the like, in a specific implementation process, the device may further include other components necessary for implementing normal running. In addition, a person skilled in the art may understand that the foregoing device may include only components necessary for implementing the solutions of this application, and does not need to include all the components shown in the figure.

Through the foregoing descriptions of the implementations, a person skilled in the art can clearly learn that this application may be implemented by using software in combination with a necessary universal hardware platform. Based on such an understanding, the technical solution of this application, in essence, or a part contributing to the related art may be embodied in a form of a computer program product. The computer program product may be stored in a storage medium, for example, a ROM/RAM, a magnetic disk, or a compact disc, including several instructions for causing a computer device (which may be a personal computer, a server, or a network device) to perform the method according to the embodiments or some parts of the embodiments of this application.

The technical solutions provided in this application are described above in detail. Although the principles and the implementations of this application are described by using specific examples in this specification, the descriptions of the foregoing embodiments are merely used for helping understand the method of this application and the core idea of the method. Meanwhile, a person of ordinary skill in the art may make modifications to the specific implementations and the application scope based on the idea of this application. In conclusion, the content of this specification should not be construed as a limitation on this application.

The various embodiments described above can be combined to provide further embodiments. Aspects of the embodiments can be modified, if necessary to employ concepts of the various embodiments to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. An image classification method, comprising:
obtaining an image;
performing feature extraction on the image to obtain a feature representation of the image;
performing, by using the feature representation of the image, cross-attention processing on an initial representation of a plurality of cluster centers to obtain a plurality of cluster center representations;
performing decoding by using the feature representation of the image and the plurality of cluster center representations to obtain a category assignment matrix; and
performing classification by using the plurality of cluster center representations and the category assignment matrix to obtain a classification result indicating whether the image belongs to a target category.

2. The method according to claim 1, further comprising:
segmenting the image by using the category assignment matrix to obtain an image area of a category, wherein the category comprises the target category.

3. The method according to claim 1, wherein the performing feature extraction on the image comprises:
performing feature extraction on the image to obtain a feature representation of an element token at each of a plurality of resolutions, and determining a feature representation of the element token at a highest resolution as the feature representation of the image.

4. The method according to claim 3, wherein the performing, by using the feature representation of the image, cross-attention processing on an initial representation of a plurality of cluster centers comprises:
obtaining a query matrix by using the initial representation of the plurality of cluster centers;
inputting the query matrix to a multilayer concatenated transformer network, wherein each layer of the transformer network corresponds to a resolution of an ascending order of resolutions;
obtaining, by each layer of the transformer network, a key matrix and a value matrix by using a feature representation of each element token at a corresponding resolution;
performing cross-attention processing on the query matrix input to the layer of the transformer network to obtain a query matrix output by the layer of the transformer network; and
obtaining the plurality of cluster center representations by using a query matrix output by a last layer of transformer network.

5. The method according to claim 1, wherein the performing classification by using the plurality of cluster center representations and the category assignment matrix comprises:
performing averaging on the plurality of cluster center representations to obtain a cluster-averaged representation;
performing pooling on the category assignment matrix to obtain a cluster-pooled representation; and
integrating the cluster-averaged representation and the cluster-pooled representation to obtain an integrated feature representation, and performing classification by using the integrated feature representation to obtain the classification result.

6. The method according to claim 1, further comprising:
obtaining training data comprising a plurality of training samples, wherein the training sample comprises an image sample and a label indicating whether the image sample belongs to the target category;
training an image classification model by using the training data, wherein the image classification model comprises a feature extraction network, a first decoding network, a second decoding network, and a classification network, and the training the image classification model includes:
performing, by the feature extraction network, feature extraction on the image sample to obtain a sample feature representation of the image sample;
performing, by the first decoding network, cross-attention processing on an initial representation of a plurality of sample cluster centers by using the sample feature representation of the image sample to obtain a plurality of sample cluster center representations;
performing, by the second decoding network, decoding by using the sample feature representation of the image sample and the plurality of sample cluster center representations to obtain a sample category assignment matrix; and
performing, by the classification network, classification by using the plurality of sample cluster center representations and the sample category assignment matrix to obtain a sample classification result indicating whether the image sample belongs to the target category; and
wherein a target of the training comprises minimizing a difference between the sample classification result and a corresponding label of the image sample.

7. The method according to claim 6, wherein the training sample further comprises an area mask of a sample category marked on the image sample, and the image classification model further comprises a segmentation network;
the segmentation network segments the image sample by using the sample category assignment matrix to obtain an image area of the sample category, wherein the sample category comprises the target category; and
the target of the training further comprises minimizing a difference between the image area of the sample category and the area mask of the sample category.

8. The method according to claim 6, wherein the performing feature extraction on the image sample comprises:
performing feature extraction on the image sample to sequentially obtain a sample feature representation of each element token at a plurality of resolutions, and determining a sample feature representation of each element token at a highest resolution as the sample feature representation of the image sample; and
the first decoding network comprises a multilayer concatenated transformer network, the initial representation of the plurality of sample cluster centers is determined as a sample query matrix input to a first layer of the transformer network, each layer of the transformer network is in one-to-one correspondence with a resolution of the plurality of resolutions based on an ascending order of the plurality of resolutions, each layer of the transformer network obtains a sample key matrix and a sample value matrix by using a sample feature representation of each element token at a corresponding resolution, and the cross-attention processing is performed on the query matrix input to the layer of the transformer network to obtain a sample query matrix output by the layer of the transformer network; and the plurality of sample cluster center representations are obtained by using a sample query matrix output by a last layer of the transformer network.

9. The method according to claim 6, wherein the performing classification by using the plurality of sample cluster center representations and the sample category assignment matrix comprises:
performing, by the classification network, averaging on the plurality of sample cluster center representations to obtain a sample cluster-averaged representation;
performing pooling on the sample category assignment matrix to obtain a sample cluster-pooled representation; and
cluster-pooled representation to obtain an integrated sample feature representation, and performing classification by using the integrated sample feature representation to obtain the sample classification result.

10. A computing system including one or more processors and one or more storage devices, the one or more storage devices, individually or collectively, having computer executable instructions stored thereon, the computer executable instructions, when executed by the one or more processors, enabling the one or more processors to, individually or collectively, implement actions comprising:
obtaining an image;
performing feature extraction on the image to obtain a feature representation of the image;
performing, by using the feature representation of the image, cross-attention processing on an initial representation of a plurality of cluster centers to obtain a plurality of cluster center representations;
performing decoding by using the feature representation of the image and the plurality of cluster center representations to obtain a category assignment matrix; and
performing classification by using the plurality of cluster center representations and the category assignment matrix to obtain a classification result indicating whether the image belongs to a target category.

11. The computing system according to claim 10, wherein the actions further comprise:
segmenting the image by using the category assignment matrix to obtain an image area of a category, wherein the category comprises the target category.

12. The computing system according to claim 10, wherein the performing feature extraction on the image comprises:
performing feature extraction on the image to obtain a feature representation of an element token at each of a plurality of resolutions, and determining a feature representation of the element token at a highest resolution as the feature representation of the image.

13. The computing system according to claim 12, wherein the performing, by using the feature representation of the image, cross-attention processing on an initial representation of a plurality of cluster centers comprises:
obtaining a query matrix by using the initial representation of the plurality of cluster centers;
inputting the query matrix to a multilayer concatenated transformer network, wherein each layer of the transformer network corresponds to a resolution of an ascending order of resolutions;
obtaining, by each layer of the transformer network, a key matrix and a value matrix by using a feature representation of each element token at a corresponding resolution;
performing cross-attention processing on the query matrix input to the layer of the transformer network to obtain a query matrix output by the layer of the transformer network; and
obtaining the plurality of cluster center representations by using a query matrix output by a last layer of transformer network.

14. The computing system according to claim 10, wherein the performing classification by using the plurality of cluster center representations and the category assignment matrix comprises:
performing averaging on the plurality of cluster center representations to obtain a cluster-averaged representation;
performing pooling on the category assignment matrix to obtain a cluster-pooled representation; and
integrating the cluster-averaged representation and the cluster-pooled representation to obtain an integrated feature representation, and performing classification by using the integrated feature representation to obtain the classification result.

15. The computing system according to claim 10, wherein the actions further comprise:
obtaining training data comprising a plurality of training samples, wherein the training sample comprises an image sample and a label indicating whether the image sample belongs to the target category;
training an image classification model by using the training data, wherein the image classification model includes a feature extraction network, a first decoding network, a second decoding network, and a classification network, and the training the image classification model includes:
performing, by the feature extraction network, feature extraction on the image sample to obtain a sample feature representation of the image sample;
performing, by the first decoding network, cross-attention processing on an initial representation of a plurality of sample cluster centers by using the sample feature representation of the image sample to obtain a plurality of sample cluster center representations;
performing, by the second decoding network, decoding by using the sample feature representation of the image sample and the plurality of sample cluster center representations to obtain a sample category assignment matrix; and
performing, by the classification network, classification by using the plurality of sample cluster center representations and the sample category assignment matrix to obtain a sample classification result indicating whether the image sample belongs to the target category; and
wherein a target of the training comprises minimizing a difference between the sample classification result and a corresponding label of the image sample.

16. The computing system according to claim 15, wherein the training sample further comprises an area mask of a sample category marked on the image sample, and the image classification model further comprises a segmentation network;
the segmentation network segments the image sample by using the sample category assignment matrix to obtain an image area of the sample category, wherein the sample category comprises the target category; and
the target of the training further comprises minimizing a difference between the image area of the sample category and the area mask of the sample category.

17. The computing system according to claim 15, wherein the performing feature extraction on the image sample comprises:
performing feature extraction on the image sample to sequentially obtain a sample feature representation of each element token at a plurality of resolutions, and determining a sample feature representation of each element token at a highest resolution as the sample feature representation of the image sample; and
the first decoding network comprises a multilayer concatenated transformer network, the initial representation of the plurality of sample cluster centers is determined as a sample query matrix input to a first layer of the transformer network, each layer of the transformer network is in one-to-one correspondence with a resolution of the plurality of resolutions based on an ascending order of the plurality of resolutions, each layer of the transformer network obtains a sample key matrix and a sample value matrix by using a sample feature representation of each element token at a corresponding resolution, and the cross-attention processing is performed on the query matrix input to the layer of the transformer network to obtain a sample query matrix output by the layer of the transformer network; and the plurality of sample cluster center representations are obtained by using a sample query matrix output by a last layer of the transformer network.

18. The computing system according to claim 15, wherein the performing classification by using the plurality of sample cluster center representations and the sample category assignment matrix comprises:
performing, by the classification network, averaging on the plurality of sample cluster center representations to obtain a sample cluster-averaged representation;
performing pooling on the sample category assignment matrix to obtain a sample cluster-pooled representation; and
cluster-pooled representation to obtain an integrated sample feature representation, and performing classification by using the integrated sample feature representation to obtain the sample classification result.

19. A non-transitory storage device having computer executable instructions stored thereon, the computer executable instructions, when executed by one or more processors, enabling the one or more processors to, individually or collectively, implement actions comprising:
obtaining an image;
performing feature extraction on the image to obtain a feature representation of the image;
performing, by using the feature representation of the image, cross-attention processing on an initial representation of a plurality of cluster centers to obtain a plurality of cluster center representations;
performing decoding by using the feature representation of the image and the plurality of cluster center representations to obtain a category assignment matrix; and
performing classification by using the plurality of cluster center representations and the category assignment matrix to obtain a classification result indicating whether the image belongs to a target category.

20. The non-transitory storage device according to claim 19, wherein the actions further comprise:
obtaining training data comprising a plurality of training samples, wherein the training sample comprises an image sample and a label indicating whether the image sample belongs to the target category;
training an image classification model by using the training data, wherein the image classification model includes a feature extraction network, a first decoding network, a second decoding network, and a classification network, and the training the image classification model includes:
performing by the feature extraction network, feature extraction on the image sample to obtain a sample feature representation of the image sample;
performing, by the first decoding network, cross-attention processing on an initial representation of a plurality of sample cluster centers by using the sample feature representation of the image sample to obtain a plurality of sample cluster center representations;
performing, by the second decoding network, decoding by using the sample feature representation of the image sample and the plurality of sample cluster center representations to obtain a sample category assignment matrix; and
performing, by the classification network, classification by using the plurality of sample cluster center representations and the sample category assignment matrix to obtain a sample classification result indicating whether the image sample belongs to the target category; and
wherein a target of the training comprises minimizing a difference between the sample classification result and a corresponding label of the image sample.
